SEQUENCE LISTING (5 sequences)

SEQ ID NO: 1 - protein, 143 aa, Xerocomus chrysenteron
MSYSITLRVYQTNRDRGYFSIVEKTVWHFANGGTWSEANGAHTLTQGGSGTSGVLRFLSTKGERITVAVGVHNYKRWCDVVTGLKPDETALVINPQYYNNGGRDYVREKQLAEYSVTSAIGTKVEVVYTVAEGNNLEANVIFS

SEQ ID NO: 2 - protein, 143 aa, Artificial Sequence
  Variant A38C
MSYSITLRVYQTNRDRGYFSIVEKTVWHFANGGTWSECNGAHTLTQGGSGTSGVLRFLSTKGERITVAVGVHNYKRWCDVVTGLKPDETALVINPQYYNNGGRDYVREKQLAEYSVTSAIGTKVEVVYTVAEGNNLEANVIFS

SEQ ID NO: 3 - protein, 143 aa, Artificial Sequence
  Variant T12C
MSYSITLRVYQCNRDRGYFSIVEKTVWHFANGGTWSEANGAHTLTQGGSGTSGVLRFLSTKGERITVAVGVHNYKRWCDVVTGLKPDETALVINPQYYNNGGRDYVREKQLAEYSVTSAIGTKVEVVYTVAEGNNLEANVIFS

SEQ ID NO: 4 - protein, 143 aa, Artificial Sequence
  Variant T12C A38C
MSYSITLRVYQCNRDRGYFSIVEKTVWHFANGGTWSECNGAHTLTQGGSGTSGVLRFLSTKGERITVAVGVHNYKRWCDVVTGLKPDETALVINPQYYNNGGRDYVREKQLAEYSVTSAIGTKVEVVYTVAEGNNLEANVIFS

SEQ ID NO: 5 - protein, 143 aa, Xerocomus chrysenteron
MSYSITLHVYQRNPARGFFHVVEQTVWHYANGGTWSEANGALTLTQGGSGTSGVIRFLSDKGERITVAVGVHNYKRWCDVVTGLKPDQTALVINGEYYNEGKRAYAREKQLAEYSVTSAVGTKVEVVYTVAEGNNLKANVIIG

(19) United States
(12) Patent Application Publication
(10) Pub. No.: US 2015/0182631 A1
(43) Pub. Date: Jul. 2, 2015

(54) SYSTEM FOR DELIVERING LECTIN-BASED ACTIVE INGREDIENTS

(71) Applicants: Centre National De La Recherche Scientifique-CNRS, Paris (FR); Universite Paul Sabatier Toulouse III, Toulouse (FR)

(72) Inventors: Laurent Paquereau, Baziege (FR); Gregori Gross, Toulouse (FR); Caroline Ladurantie, Toulouse (FR)

(21) Appl. No.: 14/416,599

(22) PCT Filed: Jul. 19, 2013
(86) PCT No.: PCT/FR2013/051751

§ 371 Date: Jan. 22, 2015

(30) Foreign Application Priority Data
Jul. 23, 2012 (FR) 12 57125

(51) Int. Cl. A61K 47/48

(52) U.S. Cl. CPC A61K 47/48261

(57) ABSTRACT

The invention relates to the use of fungal sporocarp lectin or of a variant thereof for the delivery of an active agent to a biological target.

### TECHNICAL FIELD

This invention generally relates to a delivery system and more particularly to a lectin-based delivery system.

The invention has applications, in particular, in the treatment of cancer.

### PRIOR ART

Recent developments in the pharmaceutical field have concerned protein-based delivery systems. These protein-based platforms are very good candidates for use in the medical field. Indeed, these systems show good biocompatibility and biodegradability combined with low toxicity.

A large number of proteins have been used as a system for delivering drugs including ferritin/apoferritin, capsids of various viruses, albumin, gliadin etc. These protein delivery systems have diverse forms such as microspheres, nanoparticles, hydrogels, films and protein cages.

Protein-based delivery systems and especially protein cages appear to be a promising delivery system which makes it possible to avoid certain disadvantages of polymer-based delivery systems thanks to their uniform size, their bioavailability and their biodegradability (Maham et al., 2009).

There is therefore a need for new protein-based delivery systems. In addition to their bioavailability and biodegradability properties, these delivery systems must be able to be manufactured in a simple and reproducible manner and in large quantities.

In addition, these delivery systems must be designed to deliver therapeutic agents specifically to the cells to be treated in order to limit the side effects linked to the treatment.

Therefore, the delivery systems must confine the therapeutic agents inside their protein cage and release them once the target to be treated has been reached.

Finally, delivery systems must be characterised easily and their safety must be demonstrated.

### SUMMARY OF THE INVENTION

The family of fungal sporocarp lectins, also called group-2 mushroom lectins, is a family of fungal lectins having both a sequence and structure homology.

The sequence homologies of the members of this family of lectin are largely described in Birck C et al. (2004), Trigueros et al. (2003) and Khan et al. (2011).

In addition, the analysis of the three-dimensional structures of certain members has shown a similarity in the structure. As such, it has been shown that Xerocomus chrysenteron lectin (XCL) (Birck C et al. (2004)), Agaricus bisporus lectin (ABL), (Carrizo M. et al (2004)) and Boletus edulis lectin (BEL) (Michele Bovi et al. ((2011)) all three form a tetramer having a central cavity.

This family of fungal sporocarp lectins includes, in particular, Agaricus bisporus lectin (ABL), Arthrobotrys oligospora lectin (AOL), Boletus edulis lectin (BEL), Gibberella zeae lectin (GZL), Xerocomus chrysenteron lectin (XCL), Pleurotus cornucopiae lectin (PCL) and Paxillus involutus lectin (PIL) (Birck et al., 2004) (Crenshaw et al., 1995).

The inventors have succeeded in confining an active agent within the cavity of fungal sporocarp lectin multimer and have shown that the active agent confined as such was then able to be delivered to a biological target.

The use of fungal sporocarp lectin as a confinement complex makes it possible to suppress, or at least lessen, all or a part of the disadvantages of delivery systems of prior art.

Indeed, these lectins have an affinity and a specificity for an epithelial tumour marker. Among the members of this family, there is a lectin, ABL, present in the mushroom Agaricus Bisporus, i.e. the white mushroom. This mushroom is one of the most consumed in the world. A toxicity or an allergenicity of this protein with regards to the human species can therefore be excluded, a priori.

The lectins of this family and their variants are easily produced recombinantly and excessively substantial levels of production and purification (g/litre) can therefore be reached.

These lectins are particularly stable over time (several months at 4° C., several weeks at ambient temperature) and resist harsh physical-chemical conditions (SDS, temperature, ionic strength) very well.

Finally, in addition to their capacity to reversibly confine an active agent and its low toxicity, these lectins form a multimer with a cavity that can contain an active agent without requiring a covalent bond between the multimer and the active agent that it contains.

A first aspect of the invention consequently relates to the use of a fungal sporocarp lectin or of a variant of a fungal sporocarp lectin for the delivery of an active agent which is a therapeutic agent or a diagnostic agent to a biological target.

In a second aspect, the invention also relates to a complex comprising an active agent which is a therapeutic agent or a diagnostic agent and a fungal sporocarp lectin or of a variant of a fungal sporocarp lectin.

While seeking to improve the performance of these lectins in the confinement of active agents, the inventors discovered that certain variants of XCL, a lectin particularly suited for its use as a delivery system, had an improved confinement capacity while still retaining their capacity to release the active agent once the target is reached. The invention relates to this type of variant, more precisely a variant of XCL having an amino acid sequence chosen from the group consisting of SEQ ID NO: 2, SEQ ID NO: 3 and SEQ ID NO: 4.

Another aspect of the invention also provides for:
a pharmaceutical composition comprising a complex according to the invention and a pharmaceutically acceptable excipient,
a fungal sporocarp lectin, a variant thereof or a complex according to the invention for its use in a method of treating the human or animal body and, in particular, in a method for treating cancer.

### DETAILED DESCRIPTION

### Use of Fungal Sporocarp Lectin for the Delivery of an Active Agent

The inventors have succeeded in controlling the structuring of a multimer of certain fungal sporocarp lectins in such a way that an active agent can be inserted in a stable manner into their cavity and released once the multimer-active agent complex has reached a given biological target.

The invention therefore relates to the use of a fungal sporocarp lectin or of a variant of a fungal sporocarp lectin for the delivery of an active agent which is a therapeutic agent or a diagnostic agent to a biological target.

The term “therapeutic agent” refers to an agent that has a pharmacological activity or a benefit on health when it is administered in a therapeutically effective amount.

In a preferred embodiment, the therapeutic agent is a chemotherapeutic agent.

The chemotherapeutic agent can be a cytotoxic chemotherapeutic agent such as, for example, an agent that damages DNA, an antimetabolite, an antimitotic or a Vinca alkaloid (Cancer immunotherapy: immune suppression and tumor growth, George C. et al.) (Chemotherapy at Dorland's Medical Dictionary)).

Agents that damage DNA can be alkylating agents such as cyclophosphamide, chlorambucil, chlormethine, busulfan, treosulfan and thiotepa, topoisomerase inhibitors such as camptothecin, irinotecan and topotecan or amsacrine, etoposide, etoposide phosphate and teniposide or platinum-based compounds such as cisplatin, carboplatin, oxaliplatin.

Examples of anti-tumour antibiotics are anthracyclins such as doxorubicin, epirubicin, idarubicin, mitoxantrone, valrubicin or mitomycin.

Examples of anti-metabolites are folate antagonists such as methotrexate, purine antagonists such as fludarabine and pyrimidine antagonists such as 5-fluorouracil.

Examples of antimitotics are taxanes such as paclitaxel, and docetaxel.

Examples of Vinca alkaloids are vincristine, vinblastine, vinorelbine and vindesine.

The term “diagnostic agent” refers to an agent that when it is administered in an effective amount makes it possible to identify if a subject is afflicted with or is susceptible of developing a given pathology.

In an embodiment, the diagnostic agent can be a radioactive agent or a fluorescent agent.

The diagnostic agent can for example include a radioisotope of iodine (I), such as 123I, 125I, 131I, etc., of barium (Ba), of gadolinium (Gd), of technetium (Tc) including 99Tc, of phosphorus (P) including 31P, of iron (Fe), of manganese (Mn), of thallium (TI), chromium (Cr), including 51Cr, of carbon (C) including 14C or of fluorescently labelled compounds.

In a preferred embodiment, the diagnostic agent cannot or hardly be detected when it is confined in the lectin multimer and only becomes significantly detectable once it is released at the level of the biological target.

In a preferred embodiment, the active agent according to the invention is a therapeutic agent.

The term “variant” refers herein to a protein having an amino acid sequence that has at least 80% identity with the amino acid sequence of the protein of which it is the variant and which has a capacity to confine an active agent that is substantially greater than or equal to that of the protein of which it is the variant.

Typically, the variant of a protein has at least 80%, 85%, 90%, 95%, 96%, 97%, 98% or 99% identity with the amino acid sequence of the protein of which it is the variant.

The percentage identity refers to comparisons of amino acid sequences and is determined by comparing two optimally aligned sequences on a window discriminator, in which the portion of the amino acid sequence in the window discriminator can comprise additions or deletions (i.e. differences) with respect to the reference sequence (which does not comprise any additions or deletions) for an optimum alignment of the two sequences. The percentage can be calculated by determining the number of positions where there is an identical amino acid residue in the two sequences in order to obtain the total number of positions in the window discriminator and by multiplying the result by 100 in order to reach the percentage sequence identity. Alternatively, the percentage can be calculated by determining the number of positions where there is an identical amino acid residue in the two sequences where an amino acid residue is aligned with a difference in order to reach the corresponding number of positions, by dividing the corresponding number of positions by the total number of positions in the window discriminator and by multiplying the result by 100 in order to reach the percentage sequence identity. Those skilled in the art know that there are several known algorithms for aligning two sequences. An optimum aligning of sequences for a comparison can be carried out, for example, by the Smith and Waterman local alignment algorithm, 1981, Adv. Appl. Math. 2:482, by the Needleman and Wunsch algorithm, 1970, J. Mol. Biol. 48:443, by the Pearson and Lipman similarity search method, 1988, Proc. Natl. Acad. ScL USA 85:2444, by computerised implementations of these algorithms (GAP, BESTFIT, FASTA, and TFASTA), or via visual verification (Current Protocols in Molecular Biology, F. M. Ausubel et al., eds., (1995 Supplement) (Ausubel)). Examples of algorithms that are suited for determining the percentage sequence of identity and of similarity are the BLAST and BLAST 2.0 algorithms which are described in Altschul et al., 1990, J. Mol. Biol. 215: 403-410 and Altschul et al., 1977, Nucleic Acids Res. 3389-3402, respectively. The software for conducting BLAST analyses is available at the National Center for Biotechnology Information website.

The capacity to confine an active agent can be measured according to the fluorescein method described in the examples.

In a preferred embodiment, the fungal sporocarp lectin of the invention is chosen from the group consisting of ABL, AOL, GZL, XCL, PCL, BEL and PIL or a variant thereof.

Preferably, the fungal sporocarp lectin is XCL, ABL, BEL or a variant thereof.

XCL, the lectin of Xerocomus chrysenteron, is a protein belonging to a family of fungal sporocarp lectins that has been isolated using an edible mushroom by man, Xerocomus chrysenteron.

This protein is known for its insecticide activity and has, in particular, been described by Wang M et al. (2002), Trigueros V et al. (2003) and Birck C et al. (2004).

The amino acid sequence of this protein is SEQ ID NO: 1.

XCL has XCL2 of SEQ ID NO: 5 as isoform.

Birck C et al. (2004) have shown that XCL appears, in solution, in the form of a tetrameric structure, generating at its centre a cavity of which the walls are delimited by each monomer. This same structure is found for Agaricus bisporus lectin, ABL (Carrizo M. et al (2004)) and Boletus edulis lectin, BEL (Michele Bovi et al. ((2011)).

The inventors have discovered that certain variants of XCL had optimal characteristics for use as a confinement complex.

These three preferred variants are mutants of XCL for which the threonine 12 was replaced with a cysteine and/or the alanine 38 was replaced with a cysteine. These variants improve the maintaining of the tetrameric assembly of XCL in its closed form while still being able to control its opening.

The inventors have, in particular, shown that the interchain disulphide bridge of the variant A38C provides two types of structural constraints. On the one hand, this covalent bond reduces the freedom of movement of the loops closing the access to the cavity of the protein cage which prevents the leakage of the confined active agent. On the other hand, the adding of bonds between the monomers implies that the dissociation of the oxidised variant can take place only if the four interfaces are broken. This variant therefore has a highly stabilised tetrameric structure.

The invention consequently relates to a variant of XCL that has an amino acid sequence chosen from the group consisting of SEQ ID NO: 2, SEQ ID NO: 3 and SEQ ID NO: 4.

TABLE 1
Summary of the sequences

| | SEQ ID | |
|---|---|---|
| Protein | NO | Amino acid sequence |
| XCL | SEQ ID | MSYSITLRVYQTNRDRGYFSIVEKTVW |
| | NO: 1 | HFANGGTWSEANGAHTLTQGGSGTSGV |
| | | LRFLSTKGERITVAVGVHNYKRWCDVV |
| | | TGLKPDETALVINPQYYNNGGRDYVRE |
| | | KQLAEYSVTSAIGTKVEVVYTVAEGNN |
| | | LEANVIFS |
| XCL variant: | SEQ ID | MSYSITLRVYQTNRDRGYFSIVEKTVW |
| A38C | NO: 2 | HFANGGTWSECNGAHTLTQGGSGTSGV |
| | | LRFLSTKGERITVAVGVHNYKRWCDVV |
| | | TGLKPDETALVINPQYYNNGGRDYVRE |
| | | KQLAEYSVTSAIGTKVEVVYTVAEGNN |
| | | LEANVIFS |
| XCL variant: | SEQ ID | MSYSITLRVYQCNRDRGYFSIVEKTVW |
| T12C | NO: 3 | HFANGGTWSEANGAHTLTQGGSGTSGV |
| | | LRFLSTKGERITVAVGVHNYKRWCDVV |
| | | TGLKPDETALVINPQYYNNGGRDYVRE |
| | | KQLAEYSVTSAIGTKVEVVYTVAEGNN |
| | | LEANVIFS |
| XCL variant: | SEQ ID | MSYSITLRVYQCNRDRGYFSIVEKTVW |
| T12C | NO: 4 | HFANGGTWSECNGAHTLTQGGSGTSGV |
| and A38C | | LRFLSTKGERITVAVGVHNYKRWCDVV |
| | | TGLKPDETALVINPQYYNNGGRDYVRE |
| | | KQLAEYSVTSAIGTKVEVVYTVAEGNN |
| | | LEANVIFS |
| XCL variant: | SEQ ID | MSYSITLHVYQRNPARGFFHVVEQTVW |
| XCL2 Isoform | NO: 5 | HYANGGTWSEANGALTLTQGGSGTSGV |
| | | IRFLSDKGERITVAVGVHNYKRWCDVV |
| | | TGLKPDQTALVINGEYYNEGKRAYARE |
| | | KQLAEYSVISAVGTKVEVVYTVAEGNN |
| | | LKANVIIG |

In a preferred embodiment, the invention thus relates to the use of a variant of XCL having an amino acid sequence chosen from the group consisting of SEQ ID NO: 2, SEQ ID NO: 3 and SEQ ID NO: 4 for the delivery of an active agent which is a therapeutic agent or a diagnostic agent to a biological target.

In a preferred embodiment, the variant of XCL has the amino acid sequence SEQ ID NO: 2.

In a preferred embodiment, the biological target is a cancer cell.

Indeed, most of the fungal sporocarp lectins including XCL (Damian et al. (2005)) and ABL (Milton et al. (2001)) specifically recognise the antigen Thomsen Friedenreich (TF) (Damian L et al. (2005)). The TF antigen (Galβ1-3GalNAcα/β1) or its direct precursor Tn (Gal-NAc—O-serine) are expressed in most carcinoma and in particular in bladder (Langkilde N C et al. (1992)), colorectal (Itzkowitz S H, et al (1989)), gastro-intestinal, prostate, ovarian (MacLean G D, et al. (1992)), breast (Wolf B C, et al (1989) and Carraway K L, et al. (2005)), lung (Stein R et al., (1989)), skin (Heimburg J, et al. (2006)) cancers.

On the contrary, the TF antigen is hardly or is not expressed in normal tissue (Springer G F (1984) and Cao Y et al. (1996)).

Consequently, the specificity of the lectins of the invention for TF allows for the addressing of the latter to cancer cells.

The invention also relates to a method for delivering an active agent which is a therapeutic agent or a diagnostic agent to a biological target in which said active agent and a fungal sporocarp lectin or a variant of the latter are placed into contact.

Preferably, in an embodiment of the invention, the active agent is confined in the fungal sporocarp lectin multimer or a variant of the latter.

### Complex

This invention also relates to a complex comprising an active agent which is a therapeutic agent or a diagnostic agent and a fungal sporocarp lectin or of a variant of a fungal sporocarp lectin.

The active agent is a therapeutic agent or a diagnostic agent such as defined hereinabove.

Typically, the fungal sporocarp lectin or the lectin variant of the complex according to the invention is in the form of a multimer.

Typically, the lectin multimer has an internal cavity wherein the active agent is located.

Preferably, there is no covalent bond between the lectin multimer and the active agent.

In a preferred embodiment, the multimer is a homomultimer.

In another embodiment, the multimer can be a heteromultimer. In a preferred alternative of this embodiment, the heterodimer is comprised of fungal sporocarp lectin and of a variant of this lectin.

In the preferred embodiment, the multimer of the complex according to the invention is a tetramer, more preferentially a homotetramer.

Preferably, the complex according to the invention comprises a fungal sporocarp lectin chosen from the group consisting of ABL, AOL, GZL, XCL, PCL, BEL and PIL or a variant of thereof.

In a preferred embodiment, the fungal sporocarp lectin of the complex according to the invention is XCL, ABL or a variant of thereof.

In a preferred embodiment, the fungal sporocarp lectin of the complex according to the invention is XCL or a variant of XCL.

In another preferred embodiment, the fungal sporocarp lectin of the complex according to the invention is a variant of XCL having an amino acid sequence chosen from the group consisting of SEQ ID NO: 2, SEQ ID NO: 3 and SEQ ID NO: 4.

The invention also relates to a method for manufacturing a complex according to the invention characterised in that it comprises a step of placing a fungal sporocarp lectin or of a variant of fungal sporocarp lectin in contact with an active agent which is a therapeutic or diagnostic agent.

The method for manufacturing the complex can further comprise a step of eliminating the active agent that was not confined.

The method can comprise a step of reducing a fungal sporocarp lectin or a variant of thereof before the step of placing in contact with the active agent and a step of oxidation after the step of placing in contact with the active agent.

This step allows for the creation of disulphide bridges in order to stabilise the lectin multimer, in particular for variants modified with one or several cysteines, in order to be able to generate such bridges.

### Pharmaceutical Composition

The invention also relates to a pharmaceutical composition comprising a complex according to the invention and a pharmaceutically acceptable excipient.

The excipients are chosen according to the pharmaceutical form and the desired method of administration from among the normally used excipients.

The composition according to the invention can for example be administered by injection, by spraying or by mouth.

The pharmaceutical composition according to the invention can for example be in the form of a liquid, solid, gel or lyophilisate.

In these compositions, the active agent is advantageously present in physiologically effective doses.

Preferably, these pharmaceutical compositions are intended for non-systemic administration, for example enterally.

### Method of Treatment

This invention also relates to a complex according to the invention for its use in a method for treating the human or animal body.

The terms “treatment or treat” both refer to a curative treatment and preventive or prophylactic measures for preventing or slowing down the disease or pathological state targeted.

The subjects needing treatment include subjects already afflicted by the disease as well as subjects susceptible of having the disease or in whom the disease must be prevented.

Thus, the subject to be treated may have been diagnosed as having the disease or can be predisposed or susceptible of having the disease.

The invention also relates to a method of treating a subject comprising the administration to said subject of a therapeutically effective amount of a complex of the invention.

The invention also relates to the use of a complex according to the invention for manufacturing a drug.

It is known that certain lectins of this family penetrate effectively into epithelial carcinoma cells. Indeed, after bonding to the cell surface, the lectin is internalised via the clathrin-dependent pathway and is transported into late endosomes or the lysosomes of these cells (Francis F. et al, 2003). The acidic and protease conditions of these compartments cause the release of the active agent contained in the lectin multimer.

The invention relates to a complex according to the invention for its use in a method for treating cancer.

In an embodiment, the cancer is chosen from the group consisting of bladder cancer, colorectal cancer, gastro-intestinal cancer, prostate cancer, ovarian cancer, breast cancer, melanoma and lung cancer.

Preferably, the cancer is chosen from the group consisting of colorectal cancer, bladder cancer, gastro-intestinal cancer and ovarian cancer.

The invention also relates to a method for treating a cancer in a subject comprising the administration to said subject of a therapeutically effective amount of a complex according to the invention.

The invention also relates to the use of a complex according to the invention for manufacturing a drug intended to treat cancer.

The invention also relates to a method for administering an active agent to a subject comprising the step of administering to said subject a complex according to the invention.

The administration can in particular be done by the mouth or via injection.

### BRIEF DESCRIPTION OF THE DRAWINGS

Other characteristics and advantages of the invention shall further appear when reading the following description. The latter is purely for the purposes of information and must be read with regards to the annexed drawings wherein:

FIG. 1 shows the minimum and maximum distances between two ε-amino groups exposed to the surface of XCL and belonging to different protein subunits;

FIG. 2 shows the emission spectra of FITC-XCL and of RITC-XCL. A—Continuous curves: Emission spectrum of the FITC-XCL (λmax 520 nm) and of the RITC-XCL (λmax 580 nm); Discontinuous curves: deconvolution of the emission spectrum obtained in (B). B—Continuous curve: digital adding of the spectra obtained in (A) for the FITC-XCL and the RITC-XCL; Discontinuous curve: emission spectrum obtained by mixing FITC-XCL and RITC-XCL;

FIG. 3 shows the emission spectra of FITC-A38C and of RITC-A38C; A—Continuous curves: Emission spectrum of the FITC-A38C (λmax 520 nm) and of the RITC-A38C (λmax 580 nm); Discontinuous curves: deconvolution of the emission spectrum obtained in (B). B—Continuous curve: digital adding of the spectra obtained in (A) for the FITC-A38C and the RITC-A38C; Discontinuous curve: emission spectrum obtained by mixing FITC-A38C and RITC-A38C;

FIG. 4 shows the change in the 580 nm/520 nm intensity ratio according to the concentration in protein XCL.

FIG. 5 shows the variation in the elution volumes of the exclusion chromatography according to the concentrations of XCL;

FIG. 6 shows the kinetics of the exchange by measuring the FRET at 580 nm for XCL;

FIG. 7 shows the confinement of fluorescein in XCL and the reduced and non-reduced variant A38C;

FIG. 8 shows the relative intensities of fluorescence of the Trp of XCL at 346 and 330 nm according to the temperature;

FIG. 9 shows the DOSY experiment.

### EXAMPLES

### Equipment and Methods

### Preparation of Proteins

All of the mutants were carried out using the Quickchange mutagenesis kit (Stratagene). The mutants A38C and T12C of XCL were constructed using the cDNA of the native XCL protein. The proteins were produced and purified as described in literature (Trigueros et al.. (2003)). An additional step of purification was carried out on size exclusion chromatography (Sephacryl S300) balanced with 50 mM of phosphate buffer, 100 mM NaCl, pH7.2 at a flow rate of 3 ml·mn⁻¹. The purified proteins were finally concentrated at 60 μM on a Vivaspin 15R 30,000 MWCO column (Sartorius stedim). The mutant A38C was oxidised by an incubation for 8h in 50 mM of phosphate buffer, 100 mM NaCl, pH 8.5 and 10 mM of oxidised glutathione (GSSG). The excess glutathione was eliminated on PD-10 sephadex G-25M column (GE Healthcare), balanced with 50 mM of phosphate buffer, 100 mM NaCl, pH7.2.

Labelling XCL and A38C Proteins with FITC and with RITC

XCL or A38C (30 μM) are incubated at 25° C. for 1 h under stirring in a buffered medium composed of a phosphate buffer (50 mM, pH 9) and either FITC (1 mM) or RITC (1 mM). The reaction is stopped by adding Tris-HCl (10 mM, pH 9). The labelled proteins are purified on PD-10 sephadex G-25M columns (GE Healthcare) balanced beforehand with 50 mM of phosphate buffer (pH 8.5). The stoechiometry of the labelling is determined by spectrophotometry at pH 8.5. The concentration of fluorescent groupings linked to the proteins is calculated with the following extinction coefficients: [494 nm=77,000 cm⁻¹ M⁻¹ for FITC and ε560 nm=85,000 cm⁻¹ M⁻¹ for RITC. The concentrations of XCL and of A38C after the labelling are calculated with ε278 nm=120,000 cm⁻¹ M⁻¹, and by taking the absorption of the fluorescent probes at this wavelength into account.

### FRET Experiments

### Balanced FRET

Fluorescence energy transfer was used to reveal the exchange of the XCL monomers. The exchange reaction is initiated by mixing the same volume of XCL-RITC (1 μM or only the buffer as a reference) and of XCL-FITC (1 μM) at 25° C. in 50 mM of phosphate buffer, 100 mM NaCl, pH 8.5. After one hour of incubation, the emission spectrum of the sample excited at 490 nm is recorded by spectrofluorimetry (Photon Technology International QM-4). The same experiments were carried out with A38C-RITC and A38C-FITC.

### FRET Kinetics

The speed of the exchange of subunits was measured by the same technique. The reaction is initiated by mixing the same volume of XCL-RITC (1 μM) and of XCL-FITC (1 μM) at 25° C. in 50 mM of phosphate buffer, 100 mM NaCl, pH 8.5. The excitation is carried out at 468 nm and the fluorescence emission at 580 nm is recorded every 0.5 seconds. The fluorescence intensity is normalised by using the equation:

$${F}_{norm} ( t ) = \frac{F ( t ) - {F}_{0}}{{F}_{∞} - {F}_{0}}$$

where F(t) represents the experimental fluorescence in the time considered, F₀ the value of the initial fluorescence, and F_{∞} the value of the final fluorescence when the state of equilibrium is reached. The kinetic data was modelled with the GOSA software (Czaplicki et al. (2006)). The best model was obtained for a double exponential of equation:

F=1−[a×e^{-k}^{₁}^{t}+(1−a)×e^{-k}^{₂}^{t}]

where:
a represents the proportion of molecules at slow dissociation, k1 the kinetic constant of the slow dissociation and k2 the kinetic constant of the fast dissociation.

### Size Exclusion Chromatography

The size exclusion chromatography experiments were carried out on a GE-Healthcare Superose 12 PC 3.2/30 column. The columns are pre-balanced in the buffer tested, namely phosphate buffer 50 mM, 100 mM NaCl, pH7.2 or pH 4.4 in the absence or in the presence of 0.003% of SDS. The flow rate is 0.5 mL/min. The BSA and the RNase A are used as a size marker.

### Confinement

The confinement experiments are carried out by incubating the wild-type protein (XCL) or the mutants (A38C) with the molecule to be tested. In order to carry out the control experiments with the mutant A38C, an oxidation step is carried out in the presence of GSSG in a phosphate buffer 50 mM, 100 mM NaCl, pH 7.2, OVN. The GSSG is then eliminated on a PD-10 sephadex G-25M column (GE Healthcare) pre-balanced in a phosphate buffer 50 mM, 100 mM NaCl, pH 8.5. The eluate is concentred at 1 ml on Vivaspin 15R 30,000 MWCO (Sartorius stedim). A batch of A38C is reduced to TCEP (under argon) with adding of NaOH for a final pH of 8.5. The proof of concept for the confinement was carried out with fluorescein at a concentration of 10 mM. The incubation is carried out for 1h at 25° C. in the phosphate buffer 50 mM, 100 mM NaCl. For the reduced mutant A38C another step of oxidation is carried out with GSSG, OVN. The free fluorescein is eliminated on a PD-10 sephadex G-25M column (GE Healthcare). The sample is then concentrated on Vivaspin 15R 30,000 MWCO (Sartorius stedim). The measurement of the confinement is carried out using spectrophotometry. The quantity of fluorescein is measured by using an absorption coefficient of e511 nm=75,000 cm⁻¹ M⁻¹. The concentrations of XCL and of A38C after the confinement are calculated with e278 nm=120,000 cm⁻¹. The percentage of confinement is calculated by forming the ratio of the fluorescein/protein concentrations.

### Results:

Preceding studies have demonstrated that XCL was organised in the form of a tetramer having an inside cavity. The inventors have demonstrated that it was possible to confine a molecule in this cavity without a covalent bond between the molecule and XCL. In addition, the inventors have designed variants of XCL that can remain in tetrameric form for a long period of time. The inventors have also shown that the confined molecule could be released in conditions similar to those encountered in the target cells.

### Design and Production of Tetramers Stabilised by Disulphide Bridges.

In order to increase the stability of the tetrameric structure of XCL, the inventors have designed variants of XCL for which the monomers are covalently bonded.

The inventors have tested a strategy consisting in the forming of disulphide bridges between the monomers by substituting wild-type XCL amino acid residues with cysteine. In order to minimise the number of substitutions the inventors had to determine the amino acids that were close to their counterparts in the opposite monomer. As revealed by the 3D structure of XCL, two amino acids have such a property. The distance between the beta carbons of threonine 12 and its counterpart is 3.9 angstroms and that between alanine 38 and its counterpart is 3.4 angstroms. These distances are typical of the range of beta carbons of the cysteines involved in disulphide bridges (Galat et al. (2008)). Furthermore, the lateral chains of these residues are on the surface of the protein and entirely accessible to an oxidising agent. An analysis of the minimisation of energy with the WINCOTT software showed that the orientation of the lateral chain of threonine 12 and of alanine 38 and their structural environment was compatible with the formation of disulphide bridges.

By producing these molecules modified as such, the inventors want to obtain a tetrameric assembly that cannot be dissociated. The particularity of the variant A38C is based on the fact that the presence of two additional disulphide bridges cause a stabilisation of the entire tetramer. If it is considered that the XCL structure is a square in wherein each monomer is a corner, the disulphide bridges A38C will bind them diagonally. The dissociation of the oxidised tetramers A38C will be possible only if all of the interfaces are broken. A strong stabilisation of the tetramer can be considered, rendering its dissociation highly unlikely.

The clone A38C was obtained by directed mutagenesis by using the XCL plasmid as a matrix and the protein produced and purified by using the same protocol as that used to produce XCL (Trigueros et al. (2003)). A yield of 25 mg of purified protein per litre of culture was obtained. The last part of the protocol consisted in a step of oxidation for 12 hours in the presence of 10 mM of oxidised glutathione. This step is required in order to produce disulphide bridges between the monomers. The forming of these covalent bonds was verified by SDS-PAGE. Two SDS-PAGE were prepared. Beta-mercaptoethanol was added to the samples in the first gel while beta-mercaptoethanol was not added to the second gel. When XCL is heated to 95° C. 5 min before the deposit, a single band is observed with a molecular weight slightly greater than 20 kDa on the two gels. This band corresponds to the species in monomer form. On the contrary, when XCL was not heated before the deposit, a single band with a molecular weight of approximately 80 kDa is observed which implies that XCL remained in tetrameric form despite the conditions as harsh as 2% of SDS.

In reducing conditions, A38C showed exactly the same behaviour as XCL. In non-reducing conditions, when the samples were heated, 3 bands are observed. The most intense band migrates like a protein of 40 kDa i.e. like a dimeric species. These species correspond to 2 monomers bonded covalently by the creation of disulphide bridges. It represents 90% of the species in the case of A38C. Another band that represents 5% of the species and that has a molecular weight of 21 kDa is also observed. This band corresponds to the monomers coming from non-oxidised species. The third band which also represents 5% of the species, has an apparent molecular weight of 60 kDa which corresponds to the tetrameric species. All of these results show that the rational modification strategy of XCL by genetic engineering made it possible to obtain an oxidation rate of approximately 90%.

A variant T12C was produced in the same manner as A38C by directed mutagenesis by using the XCL plasmid as a matrix. By using the same protocol, 10 mg of protein per litre of culture were produced. The oxidation rate of the variant T12C was sought by using the same method by SDS-PAGE as for A38C. These rates were compared with those obtained with the variant A38C. Several oxidative conditions were tested with different concentrations of reduced glutathione. Indeed, the adding of reduced glutathione prevents the forming of incorrect bonds by allowing for the reduction of disulphide bridges which may be insufficiently stabilised by other interactions. The oxidation rate of each sample was tested by SDS-PAGE in non-reducing conditions after heating the samples 5 min at 95° C. As with A38C, 3 bands were observed for T12C. The oxidation rate obtained for T12C is therefore comparable to that obtained for A38C. However when the reduced glutathione is added to the oxidation buffer at a concentration of 10 mM a strong decrease in the oxidation rate was observed with respect to A38C. This result shows that the additional disulphide bridge of oxidised T12C is less stable than the disulphide bridge of A38C.

### Reversible Opening of the Tetramer

As previously indicated, XCL is organised in a solution in the form of a tetramer. The inventors have determined if there was a spontaneous exchange between the tetrameric forms and possible minority dimeric or monomeric forms.

Demonstration of the Spontaneous Opening and Closing of XCL.

The oligomerization balance of the XCL protein was characterised by the measurement of the appearance of resonance energy transfer of the Förster type (FRET) between two populations of XCL labelled separately either with FITC (Fluorescein IsoThioCyanate) or with RITC (Rhodamine IsoThioCyanate).

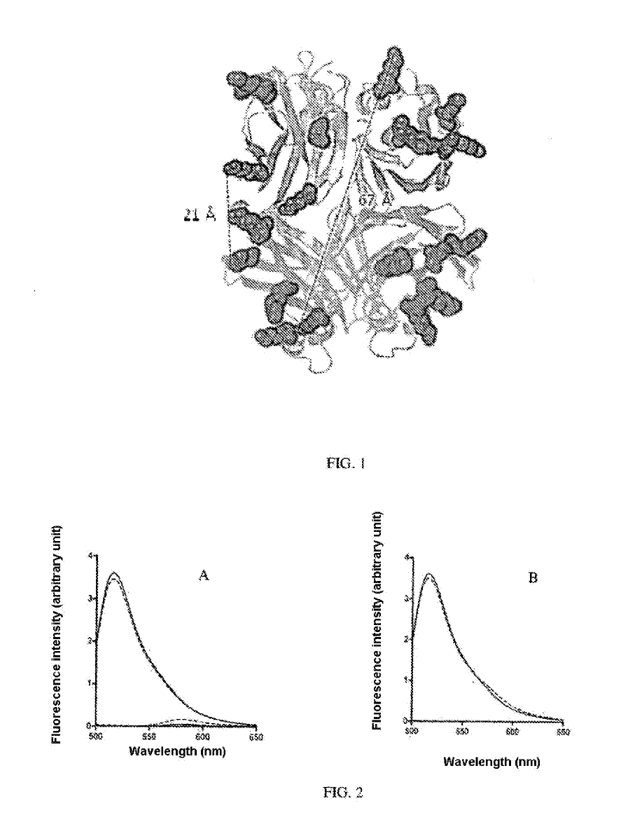

When the structure of XCL (FIG. 1) is analysed, the minimum and maximum distances between two ε-amino groups exposed to the surface and belonging to different protein subunits are respectively 21 and 67 angstroms and therefore both of them in the distance range that is compatible with the appearance of FRET.

Two samples of XCL were labelled separately with either FITC or with RITC according to the procedure described in the equipment and methods. The labelling stoechiometry is 4 mol of FTIC per mol of XCL and 0.9 mol of RITC per mol of XCL. The same stoechiometry was obtained for A38C.

The emission spectra of FITC-XCL and of RITC-XCL are shown in FIG. 2A (continuous curves). The digital adding of these spectra gives the continuous curve shown in FIG. 2B. In this same figure, is also shown the spectrum obtained by mixing the labelled protein, either by the donor or by the acceptor (dotted line curve). This spectrum was deconvoluted, which gives the spectra shown as a dotted line in FIG. 2A.

A decrease in the fluorescence intensity corresponding to the emission of fluorescein and simultaneously an increase in the fluorescence emission intensity of the rhodamine are observed, which reveals the existence of a transfer of energy between the two fluorophores.

Insofar as the transfer effectiveness depends on the donor/acceptor distance to the power of six and insofar as the dimensions of the XCL molecule are of a magnitude of the Förster distance, the appearance of this energy transfer is possible only if the FITC-XCL and RITC-XCL monomers have been redistributed and belong at the end of the experiment to the same tetramer. Consequently, access to the cavity is possible spontaneously.

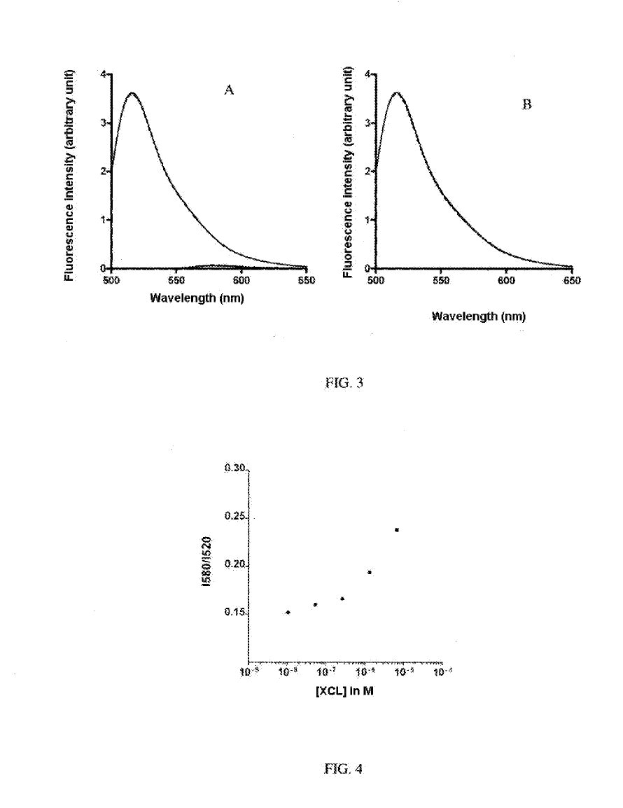

For the purposes of comparison, the same experiment was carried out with the variant A38C. This variant is covalently bonded by two additional disulphide bridges. The emission spectra of FITC-XCL and of RITC-XCL are shown in FIG. 3A (continuous curves). The digital adding of these spectra gives the continuous curve shown in FIG. 3B. A perfectly superimposable spectrum was obtained after mixing donor and acceptor labelled proteins and incubation of this mixture for 1 h at ambient temperature (dotted lines of FIG. 3B). When it is deconvoluted, the spectrum gives a spectrum represented by the dotted lines (FIG. 3A) and shows that no FRET is observed.

This experiment shows that the A38C oxidised monomers are not redistributed and remain tetrameric.

Determination of the Equilibrium Constant for the Dissociation Reaction of the Tetramer

FRET Measurements:

In the preceding paragraph, it was demonstrated that the dissociation of XCL is produced and is reversible. As such, this balance can be displaced to the dissociation and the effectiveness of the energy transfer should decrease. The inventors have taken advantage of this phenomenon for the calculation of a dissociation constant (Kd) of the tetramers of XCL.

The mixture of FITC-XCL and RITC-XCL was diluted to various concentrations and incubated for 1 hour at ambient temperature. The fluorescence spectrum was recorded with a light excitation at 468 nm. In order to substantially determine the decrease in the energy transfer and normalise the intensity of the fluorescence, a ratio I580/I520 was calculated for each concentration and taken according to the concentration in XCL (FIG. 4). A transition in the intensity of the energy transfer appears for a concentration close to the micromolar. Evidently, the titration curve is not complete as long as the saturation is not reached for the points with the strongest concentration. Due to the solubility limit of XCL and of the labelling protocol, the concentrations in protein cannot exceed 10 μM in this experiment.

Size Exclusion Chromatography Measurements:

In order to confirm the data obtained in FRET, the displacement of the balance was evaluated by gel exclusion chromatography. Bovine Serum Albumin (BSA) and ribonuclease A were used to calibrate the column. Elution volumes of 12.6 ml and of 15.5 ml were obtained respectively. At a strong concentration (here 30 μM), XCL has an elution volume of 13.2 ml close to the elution volume of BSA as expected for tetrameric species. Firstly, the existence of a balance between the tetrameric and dimeric species (or monomeric) would suggest an elution in two peaks via exclusion chromatography. However, it was shown by digital simulation that according to the association rate, the rate of dissociation, the flow rate and separation characteristics of the column, two populations of molecules that are exchanging could lead to a single peak via size exclusion chromatography (Yu et al., (2006)). The inventors carried out several injections by using variable concentrations of XCL and observed that indeed a single peak was obtained. However this peak is displaced to longer elution volumes as the concentration decreases, indicating a smaller molecular weight. When the elution volumes are taken with respect to the concentrations of XCL (FIG. 5), a transition to a concentration close to the micromolar is observed which is coherent with the results previously obtained in FRET.

Microcalorimetry Experiments

In order to accurately measure the equilibrium constant of tetramerisation and also determine the thermodynamics of the tetramerisation of XCL, the inventors used isothermal titration calorimetry (Burrows et al., (1994) Lovatt et al., (1996) Barranco-Medina et al. (2009)). In this method, a concentrated solution of XCL was diluted in a buffer, and the dissociation heat of the dimers (or of the monomers) was measured. The quantity of heat released for each injection is governed by the enthalpy exchange from the tetramer to the dimer and the tetramer-dimer equilibrium constant. The modelling of these results makes it possible to extract the equilibrium dissociation constant (Kd=4.5 μM) and a variation in enthalpy of 16.6 kilocalories per mole of tetramer.

This variation in enthalpy is in the range of values that are typically observed for an oligomeric protein of this size. However the equilibrium dissociation constant is low in comparison to those obtained for other polymeric proteins which demonstrates the remarkable stability of this protein assembly.

The convergence of these results demonstrates that XCL constantly undergoes an exchange between the dimeric form and the tetrameric form and this, even at a high protein concentration.

In order to verify that the opening and the closing of the tetramer occur at speeds that are compatible with the confinement experiments we have determined the kinetic parameters of this exchange.

Determination of the Dissociation Kinetic Constant of the Tetramer

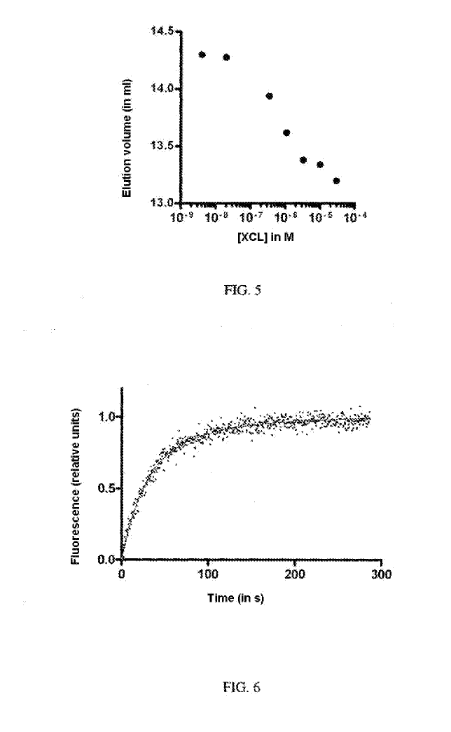

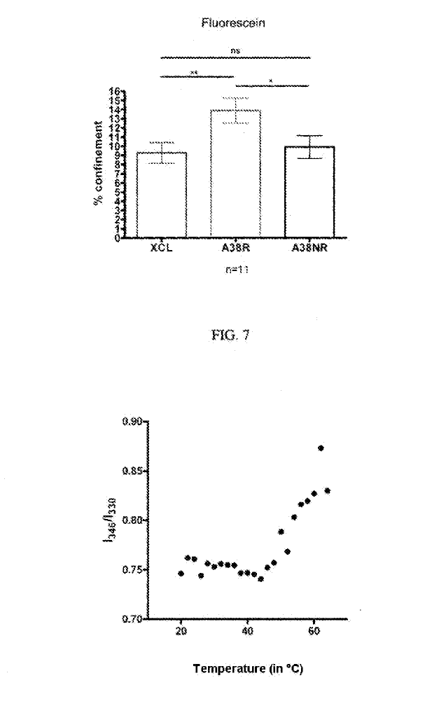

The dissociation kinetic of the tetramer was studied by mixing XCL-FITC and XCL-RITC and by measuring the fluorescence emission intensity at 580 nm as a function of time (FIG. 6). It is observed that this fluorescence intensity increases until a balance is reached more than 300 seconds of reaction, which indicates that the exchange between the subunits is slow.

The increase in the fluorescence intensity and therefore the effectiveness of the transfer between the fluorescein and the rhodamine are the result of two successive phenomena: firstly the dissociation of the tetramers into dimers and secondly their reassociation. Considering the shape of the curve (FIG. 6), no lag time is observed at the beginning of the kinetics which implies that the speed of reassociation is much faster than the speed of dissociation. It can therefore be considered that the speed of reassociation is negligible in this case. The kinetic data obtained was modelled using the GOSA software. The most suitable model for describing the phenomenon observed is a double exponential growth with two dissociation reactions that have difference speeds:

F=1−[a×e^{-k1t}+(1−a)×e^{-k2t}]

with:

a: proportion of molecules engaged in the slow reaction,

k1: kinetic constant of the slowest dissociation reaction

k2: kinetic constant of the fastest dissociation reaction

The values of the constants obtained after optimisation are:

a=0.39; k1=0.72 min-1; k2=2.52 min-1

This increase via double exponential suggests that XCL can follow two dissociation pathways. The tetramer ABCD formed by XCL can be dissociated in two different ways: into dimers AB and CD or into dimers AD and BC.

### Confining of an Agent in the Cavity

The inventors used fluorescein to validate the confining capacity of the XCL tetramer.

Fluorescein (279 Å³) has the advantage of being small and highly soluble in an aqueous solvent (it is therefore possible for this to be placed at a high concentration during confinement). In addition, it absorbs visible light at 511 nm, which makes it possible to detect it. The inventors preferred to follow the absorbance of this molecule rather than its fluorescence. Indeed, the fluorescence of a molecule depends on its chemical environment, consequently, once confined, the fluorescence output of the fluorescein could be substantially modified.

The confinement was carried out for XCL and the variant A38C.

In the case with XCL, the inventors incubated the protein (15 μM) with fluorescein (10 mM) and then separated the protein from the non-confined molecules of fluorescein by two successive gel filtration columns. Beforehand, the inventors verified that if these two columns on the fluorescein solution are carried out at 10 mM, no residual free fluorescein is detected. The fluorescein detected during the confinement is therefore necessarily linked to the protein or contained by the latter.

The protein A38C was also used to carry out the confinement, with the protocol being identical except for the fact that the protein which is initially oxidised is reduced before being put into the presence of the fluorescein solution, then in a second time oxidised for an entire night in the presence of an oxidising agent, oxidised glutathione. This protein is noted as A38R in FIG. 7.

The confinement experiment was also carried out with the protein A38C which was not reduced before being put into the presence of the fluorescein solution, which then in a second step was oxidised an entire night in the presence of an oxidising agent, oxidised glutathione. This protein is noted as A38NR in FIG. 7.

After this confinement steps and the elimination of the free fluorescein, the respective concentrations in fluorescein and in protein are determined via UV absorbance at 511 nm and at 280 nm. For XCL as well as for A38C, 11 independent experiments were carried out. Confinement rates of 9% and of 14% confinement were obtained respectively for XCL and A38R (FIG. 7).

The samples were then incubated 4 days at ambient temperature, the free fluorescein was again eliminated then the confined fluorescein was dosed. The confinement rate then remained unchanged for XCL as well as for A38C.

The fixation of the fluorescein on XCL can be linked to two types of interactions. The fluorescein can be bonded in a non-specific manner on the surface of the protein or in the cavity. The maintaining of the confinement for 4 days indicates that the fluorescein is not bonded in a labile manner, it would otherwise have dissociated after this length of time.

These confinement rates demonstrate on the one hand that an agent can be confined within the cavity and on the other hand that this cavity has non-covalent binding affinity with regards to this test molecule. Indeed, for a confinement by passive diffusion a theoretical confinement effectiveness of about 0.3% is expected. The use of A38C in oxidised form after confinement, made is possible to multiply the rate of confinement by two. The engineering strategy of the tetramer proposed by the inventors therefore makes it possible to increase the rate of confinement significantly.

Dissociation of the Complex with Acidic pH Corresponding to that of Lysosomes

In order to use XCL to transport and deliver an agent in the cells, an in-depth knowledge of the conditions of dissociation is required.

The conditions for using a confinement complex in therapy in a homeothermic organism imply that these vectors can maintain their structure in the temperature conditions of the target organism. As XCL comes from a poikilothermic organism (or heterothermic) i.e. not regulated thermally in an endogenous manner, the behaviour of the tetramer according to the temperature and in particular its behaviour beyond 45° C. was analysed.

Thermal Dissociation

Fluorescence of the Tryptophan

The thermal dissociation of XCL was followed by measuring the intrinsic fluorescence of the tryptophans. Each monomer of XCL contains 3 tryptophan residues: one in a hydrophobic cavity (Trp 77) and two located on the same interface between two monomers (Trp 27 and Trp 35). The inventors used these latter two Trp as probes for the quaternary structure of XCL, by taking advantage of their position in the structure of the tetramer. In order to increase the sensitivity of the measurement, the ratio of the fluorescence intensities 346 nm/330 nm was measured according to the temperature (FIG. 8). No modification in this ratio was observed before 50° C. which indicates that the environment of the Trp did not change up to this temperature and that the lectin remained in tetrameric form. The visual analysis of the samples after heating shows that XCL precipitates out in tetrameric form beyond 45° C. before the demasking of the tryptophans.

RMN-DOSY

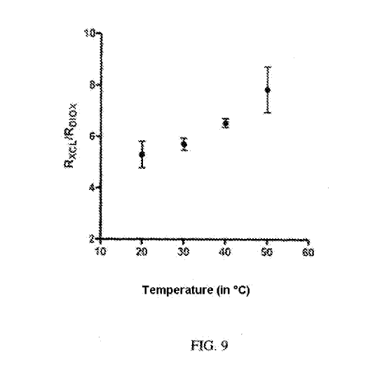

The thermal dissociation was also followed by DOSY experiments (Diffusion Ordered SpectrometrY). The ratio of the hydrodynamic radius of XCL and of the dioxane were measured according to the temperature (FIG. 9).

If the tetramer is dissociated by increasing the temperature, a decrease in this ratio is expected and therefore of the hydrodynamic radius of XCL. Inversely, the results show a linear increase in this ratio when the temperature increases. This result, which is counter-intuitive, reflects an increase in the hydrodynamic radius of XCL probably linked to the increase in the movement of the non-structured loops. In any case, no transition in the hydrodynamic radius is observed. After 50° C., the protein sample precipitates out as already described in the fluorescence experiments with tryptophan.

These two experiments demonstrate that this lectin is stable beyond 45° C. and therefore that it satisfies the thermostability criteria that are largely sufficient for use in vivo.

Chemical Dissociation

The inventors studied the behaviour of XCL in denaturing chemical conditions close to those encountered in the lysosomes. The analysis was carried out via exclusion chromatography with an acidic pH (pH 4.4). It appears that the elution peak of XCL in these denaturing conditions is displaced to greater elution volumes showing the appearance of a smaller size. When 0.003% SDS is added, the elution peak is displaced to the elution volume of the RNAse A which has a molecular weight close to the XCL monomer. The decrease in pH therefore leads to the dissociation of the tetramer into a dimer, and with a very small quantity of detergent these dimers are dissociated into monomers.

The behaviour of the oxidised mutant A38C was also analysed. At a neutral pH, A38Cox has an elution volume that is comparable to that of XCL. When the pH is decreased to 4.4, no modification in the elution volume is observed. The addition of the disulphide bridge therefore increases the stability of the protein assembly. The adding of 0.003% SDS leads to the dissociation of the tetramer into dimers, constituted of monomers bonded together by the disulphide bridge.

The lysosomes constitute subcellular degradation organelles. Their content is acidic, reducing and contains acidic hydrolases, proteases that degrade the proteins that are addressed therein. The results obtained show that in these reducing and acidic conditions, XCL or its mutant A38Cox are dissociated into dimers, allowing for the release within lysosomes of active agents confined in the cavity.

This invention was described and illustrated in this detailed description and in the Figures. This invention is not limited to the embodiments shown. Other alternatives and embodiments can be deduced and implemented by those skilled in the art when reading this description and the annexed Figures.

### REFERENCES

During this application, various references describe the state of the art of the invention. The descriptions of these references are here incorporated by reference in this description.
Barranco-Medina S, Dietz K J. Thermodynamics of 2-Cys peroxiredoxin assembly determined by isothermal titration calorimetry. Methods Enzymol. 2009; 466: 409-30. Epub 2009 Nov. 13. Birck C, Damian L, Marty-Detraves C, Lougarre A, Schulze-Briese C, Koehl P, Fournier D, Paquereau L, Samama J P. A new lectin family with structure similarity to actinoporins revealed by the crystal structure of Xerocomus chrysenteron lectin XCL. J Mol Biol. 2004 Dec. 10; 344(5): 1409-20.
Burrows S D, Doyle M L, Murphy K P, Franklin S G, White J R, Brooks I, McNulty D E, Scott M O, Knutson J R, Porter D, et al. Determination of the monomer-dimer equilibrium of interleukin-8 reveals it is a monomer at physiological concentrations. Biochemistry. 1994 Nov. 1; 33(43): 12741-5. Cao Y, Stosiek P, Springer G F, Karsten U (1996) Thomsen-Friedenreich-related carbohydrate antigens in normal adult human tissues: a systematic and comparative study. Histochem Cell Biol 106: 197-207.
Carraway K L, Ramsauer V P, Carraway C A (2005) Glycoprotein contributions to mammary gland and mammary tumor structure and function: roles of adherens junctions, ErbBs and membrane MUCs. J Cell Biochem 96: 914-926.
Carrizo M E, Capaldi S, Perduca M, Irazoqui F J, Nores G A, Monaco H L. The antineoplastic lectin of the common edible mushroom (Agaricus bisporus) has two binding sites, each specific for a different configuration at a single epimeric hydroxyl. J Biol Chem. 2005 Mar. 18; 280(11): 10614-23. Epub 2004 Dec. 13. Crenshaw, R. W., S. N. Harper, M. Moyer and L. S. Privalle, 1995. Isolation and characterization of a cDNA clone encoding a lectin gene from Agaricus bisporus. Plant Physiol., 107: 1465-1466.
Czaplicki, J., Cornelissen, G., and Halberg, F. (2006). Journal of applied biomedicine 4, 87-94
Damian L, Fournier D, Winterhalter M, Paquereau L (2005) Determination of thermodynamic parameters of Xerocomus chrysenteron lectin interactions with N-acetylgalactosamine and Thomsen-Friedenreich antigen by isothermal titration calorimetry. BMC Biochem 6: 11.
Francis F, Marty-Detraves C, Poincloux R, Baricault L, Fournier D, Paquereau L. Fungal lectin, XCL, is internalized via clathrin-dependent endocytosis and facilitates uptake of other molecules. Eur J Cell Biol. 2003 October; 82(10): 515-22.
Galat A, Gross G, Drevet P, Sato A, Ménez A. Conserved structural determinants in three-fingered protein domains. FEBS J. 2008 June; 275(12): 3207-25. Epub 2008 May 13.
Heimburg J, Yan J, Morey S, Glinskii O V, Huxley V H, Wild L, Klick R, Roy R, Glinsky V V, Rittenhouse-Olson K. Inhibition of spontaneous breast cancer metastasis by anti-Thomsen-Friedenreich antigen monoclonal antibody JAA-F11. Neoplasia. 2006 November; 8(11): 939-48.
Itzkowitz S H, Yuan M, Montgomery C K, Kjeldsen T, Takahashi H K, et al. (1989) Expression of Tn, sialosyl-Tn, and T antigens in human colon cancer. Cancer Research 49: 197-204.
Khan F. and Khan M. I. The Mushroom Lectins Show Three Types of Conserved Domain in a Bioinformatics Analysis American Journal of Biochemistry and Molecular biology 1 (4): 375-388, 2011 Langkilde N C, Wolf H, Clausen H, Orntoft T F (1992) Localization and identification of T-(Gal beta 1-3GalNAc alpha 1-O-R) and T-like antigens in experimental rat bladder cancer. J Urol 148: 1279-1284.
Lovatt M, Cooper A, Camilleri P. Energetics of cyclodextrin-induced dissociation of insulin. Eur Biophys J. 1996; 24(5): 354-7. MacLean G D, Bowen-Yacyshyn M B, Samuel J, Meikle A, Stuart G, et al. (1992) Active immunization of human ovarian cancer patients against a common carcinoma (Thomsen-Friedenreich) determinant using a synthetic carbohydrate antigen. J Immunother (1991) 11: 292-305.
Maham A, Tang Z, Wu H, Wang J, Lin Y. Protein-based nanomedicine platforms for drug delivery. Small. 2009 Aug. 3; 5(15): 1706-21.
Michele Bovi, Maria E Carrizo, Stefano Capaldi, Massimiliano Perduca, Laurent R Chiarelli, Monica Galliano, and Hugo L Monaco. Structure of a lectin with antitumoral properties in kingbolete (Boletus edulis) mushrooms, Glycobiology vol. 21 no. 8 pp. 1000-1009, 2011
Milton J D, Fernig D G, Rhodes J M: Use of a biosensor to determine the binding kinetics of five lectins for Galactosyl-Nacetylgalactosamine. Glycoconj J 2001, 18: 565-569.
Springer G F (1984) T and Tn, general carcinoma autoantigens. Science 224: 1198-1206.
Stein R, Chen S, Grossman W, Goldenberg D M (1989) Human lung carcinoma monoclonal antibody specific for the Thomsen-Friedenreich antigen. Cancer Research 49: 32-37.
Trigueros V, Lougarre A, Ali-Ahmed D, Rahbe Y, Guillot J, et al. (2003) Xerocomus chrysenteron lectin: identification of a new pesticidal protein. Biochimica Et Biophysica Acta-General Subjects 1621: 292-298.
Wang M, Trigueros V, Paquereau L, Chavant L, Fournier D (2002) Proteins as active compounds involved in insecticidal activity of mushroom fruitbodies. J Econ Entomol 95: 603-607.
Wolf B C, Salem R R, Sears H F, Horst D A, Lavin P T, et al. (1989) The expression of colorectal carcinoma-associated antigens in the normal colonic mucosa. An immunohistochemical analysis of regional distribution. Am J Pathol 135: 111-119.
Yu C M, Mun S, Wang N H (2006) Theoretical analysis of the effects of reversible dimerization in size exclusion chromatography. J Chromatogr A 1132: 99-108.

1.-10. (canceled)

11. A method for delivering an active agent that is a therapeutic agent or a diagnostic agent to a biological target, comprising a step of administering said active agent in combination with a fungal sporocarp lectin or with a variant of a fungal sporocarp lectin.

12. A complex, comprising:
an active agent which is a therapeutic agent or a diagnostic agent; and
a fungal sporocarp lectin or a variant of a fungal sporocarp lectin.

13. The complex of claim 12 wherein the active agent is a therapeutic agent.

14. The complex of claim 12 wherein the active agent is a diagnostic agent.

15. The complex of claim 12 wherein said fungal sporocarp lectin is selected from the group consisting of ABL, AOL, GZL, XCL, PCL, BEL and PIL, or wherein said variant of a fungal sporocarp lectin is a variant of a fungal sporocarp lectin that is selected from the group consisting of ABL, AOL, GZL, XCL, PCL, BEL and PIL.

16. A method for manufacturing the complex of claim 12, comprising a step of placing a fungal sporocarp lectin or a variant of a fungal sporocarp lectin in contact with an active agent which is a therapeutic agent or a diagnostic agent.

17. A variant of XCL having an amino acid sequence selected from the group consisting of SEQ ID NO: 2, SEQ ID NO: 3 and SEQ ID NO: 4.

18. A pharmaceutical composition comprising the complex of claim 12 and a pharmaceutically acceptable excipient.

19. A method for treatment of cancer, comprising administering a therapeutically effective amount of a complex to a subject in need thereof, wherein said complex comprises (i) an active agent which is a therapeutic agent or a diagnostic agent, and (ii) a fungal sporocarp lectin or a variant of a fungal sporocarp lectin.
